(19) United States
(12) Patent Application Publication
(10) Pub. No.: US 2008/0132882 A1
(43) Pub. Date: Jun. 5, 2008

(54) Orthopedic instruments with RFID

(75) Inventors: Christopher DeMaria, Glen Rock, NJ (US); Nicholas Jon LaVigna, Mountain Top, PA (US); Thies Wuestemann, Nyack, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/606,632

(22) Filed: Nov. 30, 2006

(51) Int. Cl. A61B 17/56

(52) U.S. Cl. 606/1

(57) ABSTRACT

In one aspect the present invention comprises a surgical tool. The surgical tool preferably includes a body having a cavity, and RFID electronic assembly, a first layer of first encapsulant formed around the electronic assembly and a housing insertable into the cavity. In accordance with this aspect of the present invention, a second layer of second encapsulant may be formed around the housing to hold it in place in the body cavity. In another aspect, the present invention preferably comprises an orthopedic instrument having a body which includes a cavity. Most preferably, a housing is located within the cavity, the housing preferably comprises a base member, a cap attached to the base and a glass dish attached to the cap, the glass dish being adapted to a loud frequency wave to pass through its body.

### BACKGROUND OF THE INVENTION

1. Field of the Invention

This invention is generally directed to a surgical navigation system. More particularly, this invention is directed to a system for protecting electronic circuitry used in orthopedic instruments that generally comprise a part of the navigation system from contamination, impact and the effects of sterilization.

2. Description of Related Art

Surgical navigation systems have become extremely useful tools in operating rooms. Generally, a surgical navigation system consists of one or more trackers. Each tracker is attached to a specific surgical instrument, device or implant. The system also includes a localizer. Each tracker generates one or more specific signals. These signals may be radio signals, other electromagnetic (EM) signals, light signals or ultrasonic signals. The localizer monitors the locations signals that are broadcasted. The tracker's location-identifying signals obtained by the localizer are forwarded to a processor. Based on the data contained in these signals, including their signal strength and relative phases, the processor generates data indicating the position of the tracker and the attached component.

Either prior to or at the start of the procedure, the processor is provided with data identifying the relative positions of tissue landmarks at the surgical site. The processor also has data that indicates the location of the surgical site relative to the localizer. Based on the above data, the processor provides information about the location of the surgical component attached to the tracker relative to the surgical site. Often this information is presented on a display that shows the position of the surgical component at a location within the patient. A surgical navigation system thus provides a view of the location of a surgical component at surgical site that, due to the presence of surrounding tissue, otherwise cannot be seen.

During a surgical procedure, a number of different instruments and other components are typically positioned at a surgical site. For example, during a procedure to implant an orthopedic implant, a first set of tissue cutting devices are used to gain access to the surgical site. A second set of devices are used to remove the bone and surrounding soft tissue that are to be replaced. A third set of devices shape the remaining tissue, typically the bone, so it can accept the implant. Often, trial implants are positioned at the surgical site to determine the appropriate size of implant components that should be permanently fitted to the patient. Then, the positions of the implant components themselves are tracked. Once an implant is fitted, the position of the instruments used to close the surgical site are tracked. Each of these of these instruments, implants and other components has a set of unique physical dimensions. For the surgical navigation system to accurately generate data indicating the location of a component relative to the surgical site, the system processor must have data describing component's dimensions.

U.S. patent application Ser. No. 10/214,937, filed Aug. 8, 2002, U.S. Patent Publication No. US 2003/0093103 A1, now U.S. Pat. No. ______, and incorporated herein by reference, discloses one system for providing a surgical navigation unit with data regarding the individual components applied to a surgical site. In the invention of this system, a radio frequency identification device (RFID) is attached to each component. Internal to the RFID is a memory in which data regarding the component are stored. These data identify the component and/or the physical dimensions of the component. The component also includes a coil through which the RFID is capable of exchanging signals. Prior to use, the component is attached to a handpiece. The handpiece is coupled to some a control console or station. The connection between the handpiece and the control console may be a wireless connection. Internal to the handpiece is a coil. The handpiece coil and component coil are in sufficient proximity to allow inductive signal transfer. The control console, through the handpiece, reads the data from the component RFID. These data are transferred from the console to the surgical navigation system. Based on these data, the surgical navigation system processor generates data indicating the position of the component at the surgical site.

The handpiece and the components must be sterile for use in a surgery. The handpiece and any reusable components are subject to cleaning and sterilized before use and any subsequent reuse. Generally, the most popular process utilized for sterilization is steam autoclaving. The handpiece and the components must also be protected from contamination and impaction. Thus, the electronic components contained within the orthopedic instrumentation, such as the handpiece and the components must be protected from the steam used for autoclaving, cleaning solutions and impaction.

### SUMMARY AND INVENTION

The present invention provides a surgical navigation system having components such as a reamer, a burr, a broach, a handle, or a tracker that contain electronic circuits including radio frequency identifiers (RFID). These electronic circuits need to be protected from contamination, impaction and effects of sterilization. These circuits may be housed in packages that in turn are incorporated in the components (orthopedic instruments). The protection of the electronic circuits is achieved by encapsulating them. The present invention may also be applied to rotating couplings.

Encapsulation of the electronic circuits and the packages thereof may be done using an epoxy. The assembly of RFID and electronics is placed/located into a mold cavity and uncured epoxy is injected around the assembly. The assembly of RFID, chip, antenna and electronics may also be referred to as the transponder. The epoxy is then cured. The molded transponder is placed in a cavity in the orthopedic instrument. Additional epoxy is then injected into the gap between the molded transponder and the instrument cavity and cured. The second epoxy application forms a seal between the encased electronics and instrument. Alternatively, the transponder is positioned directly in the appropriately sized cavity in the orthopedic instrument and uncured epoxy is injected around the transponder and then cured.

Yet another technique is to use polymeric-epoxy combination. This technique utilizes a polymeric housing and epoxy in combination to provide sufficient protection from contamination, impaction and sterilization. In this technique, a polymeric cap is formed via either a machining or injection molding process. A transponder is then assembled into the cap and fixed in place through the use of an adhesive/epoxy. The cap is then filled with an epoxy and cured to fully encase the transponder. This assembly is then attached to a cavity in the orthopedic instrument utilizing additional epoxy.

Additionally, to ensure a firm attachment between the transponder and the cavity in the orthopedic instrument, mechanical attachment between them may be provided via suitable methods, for example peening.

Yet another technique is to use metallic-epoxy combination. In this technique, a metallic “cap” is used in much the same way as the polymeric cap described above.

Yet another technique is to use all polymeric encapsulation. In all polymeric technique, the base and the cap of an electronic package are made from Ultem or Radel via either machining or molding. The transponder is inserted into the cap, and the cap welded to the base to form a robust seal between the two components. This assembly is then inserted into the cavity in the orthopedic instrument and attached to the orthopedic instrument such as a reamer with a metal ring via laser welding.

Yet another technique is to use ceramic-metallic combination. In this case, the ceramic material is typically glass or alumina material and the metallic components are typically stainless steel. A thin ceramic disc is bonded to the cap that is formed through conventional machining. The transponder is assembled and bonded to the ceramic disc and cap assembly, this component is then preferably resistance welded to the “base” to complete the housing.

### BRIEF DESCRIPTION OF THE DRAWINGS

The above and further objects, features and benefits of this invention are discussed in the detailed description below taken in conjunction with the accompanying drawings in which:

FIG. 1 illustratively depicts a surgical navigation system that may be used in accordance with an aspect of the present invention;

FIG. 2 is an assembly drawing that illustratively depicts packaging for components used in navigation system of FIG. 1 in accordance with an aspect of the present invention;

FIG. 3 shows the assembly of FIG. 2 mounted in a cavity in a component used in navigation system of FIG. 1 in accordance with an aspect of the present invention;

FIG. 3A shows details of the transponder assembly used in FIG. 2.

FIG. 4 shows a base used in the assembly of FIG. 2 in accordance with an aspect of the present invention;

FIG. 5 shows a cap used in the assembly of FIG. 2 in accordance with an aspect of the present invention;

FIG. 6 shows a ring used to lock the package of FIG. 2 in a component used in the surgical navigation system of FIG. 1 in accordance with an aspect of the present invention;

FIG. 7 is an assembly drawing that illustratively depicts packaging for components used in navigation system of FIG. 1 in accordance with an aspect of the present invention;

FIG. 8 is shows the assembly of FIG. 7 mounted in a cavity in a component used in navigation system of FIG. 1 in accordance with an aspect of the present invention;

FIG. 9 shows a base used in the assembly of FIG. 7 in accordance with an aspect of the present invention;

FIG. 10 shows an assembly of the base of FIG. 9 with bushings and pins in accordance with an aspect of the present invention;

FIG. 11 is a cap used in the assembly of FIG. 7 in accordance with an aspect of the present invention;

FIG. 12 shows a ceramic disc for attachment to the cap of FIG. 11 in accordance with an aspect of the present invention;

FIG. 13 is a schematic diagram illustrating the method using the polymeric-epoxy combination in accordance with an aspect of the present invention;

FIG. 14 is a schematic diagram illustrating peening in accordance with an aspect of the present invention; and

FIG. 15 is a schematic diagram illustrating use of a metallic attachment ring in accordance with an aspect of the present invention.

### DETAILED DESCRIPTION

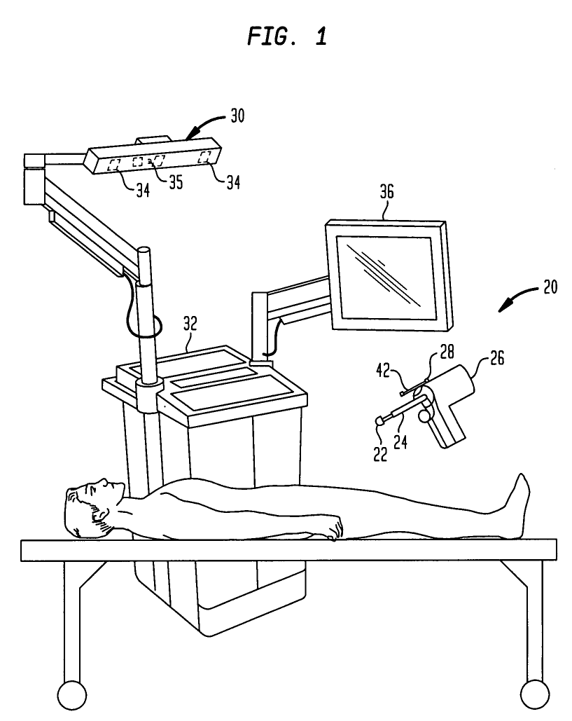

FIG. 1 depicts a surgical navigation system 20 in accordance with an aspect of the present invention that obtains data about surgical components 22 and 24 without wire connections to the components. In FIG. 1, surgical component 22 is a reamer, but may comprise a broach or burr as is discussed in further detail below. Surgical component 24 is a handle assembly. The proximal end of the handle assembly 24 is attached to a battery operated driver 26 that actuates the reamer 22. (“Proximal”, it is understood, means away from the surgical site. “Distal” means towards the surgical site.) Not shown is the battery attached to the base of the handgrip of the driver 26 that supplies the energization current for the driver. It should also be understood that the “surgical component” may be any other instrument used to cut, form or shape tissue, a trial implant or an actual implant. Sometimes, the surgical component is alternatively referred to as a “surgical implement.”

System 20 of this invention includes a tracker 28 attached to handle assembly 24. Tracker 28 broadcasts signals from one or more emitters. These signals may comprise signals having wavelengths in the electromagnetic spectrum, but are not so limited. Some trackers, for example, emit infra-red light or visible light. Other trackers emit radio frequency (RF) or electromagnetic energy. Still other trackers emit sonic energy. A localizer 30, also part of system 20, monitors the position of the tracker 28. Specifically, the localizer 30 contains one or more receivers capable of receiving the energy emitted by the tracker 28. The receivers may determine the direction from which the energy is transmitted or the strength of the received energy.

The localizer receivers send signals representative of the measurements made thereby to a processor 32, also part of system 20. Based on the determination of the different locations from which the individual tracker transmitters emit signals or the strength of the received signal or energy, the localizer 30 determines where, in three-dimensional space, the tracker 28 is positioned and the orientation of the tracker. The data representative of the location and orientation of the tracker 28, or the signals used to determine this information, are forwarded by the localizer 30 to a processor 32 also part of the system 20. Processor 32, based on the signals from the localizer 30, then generates signals representative of the position and orientation of the tracker 28. A more detailed explanation of how a surgical navigation system operates is contained in U.S. patent application Ser. No. 10/677,874, filed Oct. 2, 2003, U.S. Patent Publication No. US 2004/0073279 A1, now U.S. Pat. No. ______ and incorporated herein by reference.

As will be discussed in detail below, internal to the reamer 22 and handle assembly 24 are separate data storage devices. Each of these devices stores data that identifies the associated surgical component. These data include data that identify the physical characteristics of the component. When the reamer-handle-tracker sub-assembly is assembled, the tracker 28 reads the data in the storage devices. The tracker 28 transmits signals containing these data. A receiver 34, typically positioned in the localizer 30, reads data. The data is transmitted by the receiver 34 to the system processor 32.

Prior to the commencement of the procedure in which system 20 is employed, the processor 32 is loaded with data that identifies the location of individual body tissues and organs at the surgical site. During the procedure, the processor 32 is provided with data indicating the location of the surgical site. Based on this data, the data indicating the position and orientation of the tracker 28 and the data identifying the physical characteristics of the surgical components 22 and 24, processor 32 generates information indicating the position of the surgical components relative to the surgical site. More particularly, the processor generates information indicating the position of the surgical components relative to the body tissue at the surgical site. Typically, this information is presented visually on a display 36. The surgeon is thus able to view the position of the surgical components that otherwise cannot readily be seen.

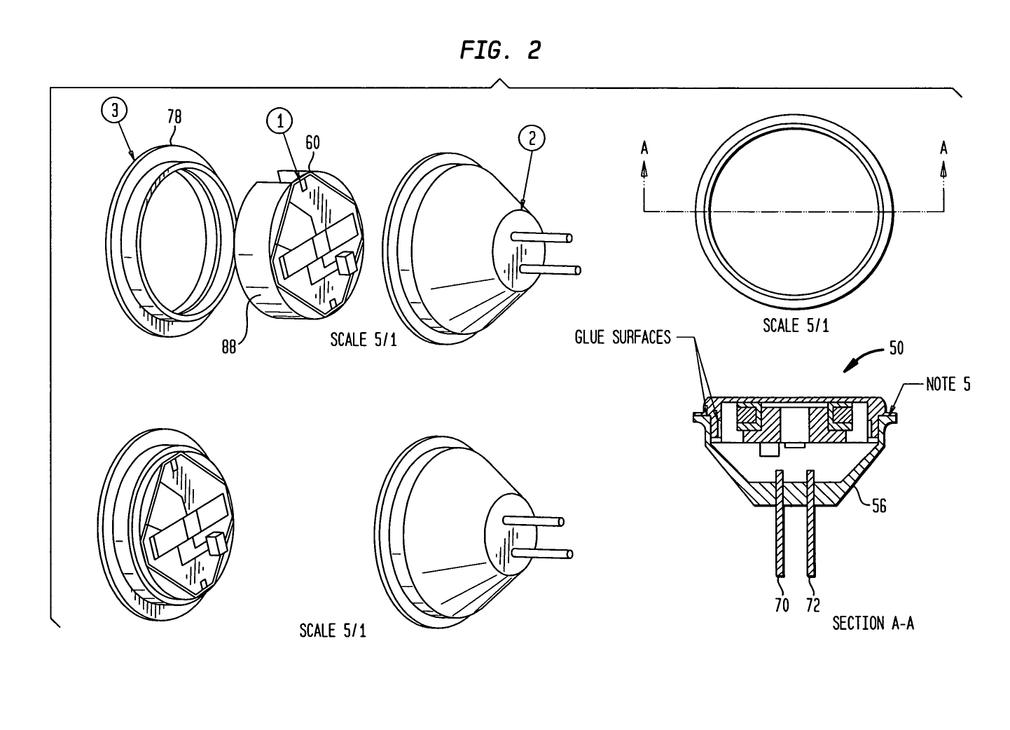

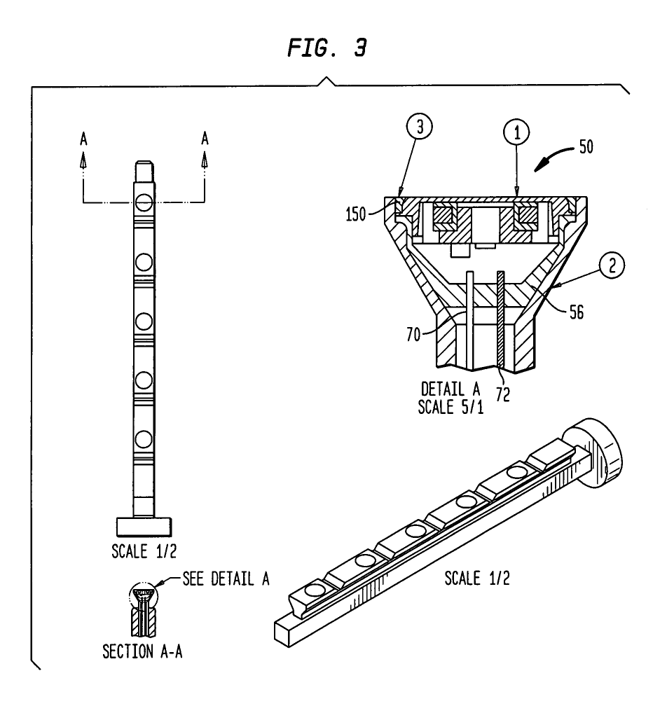

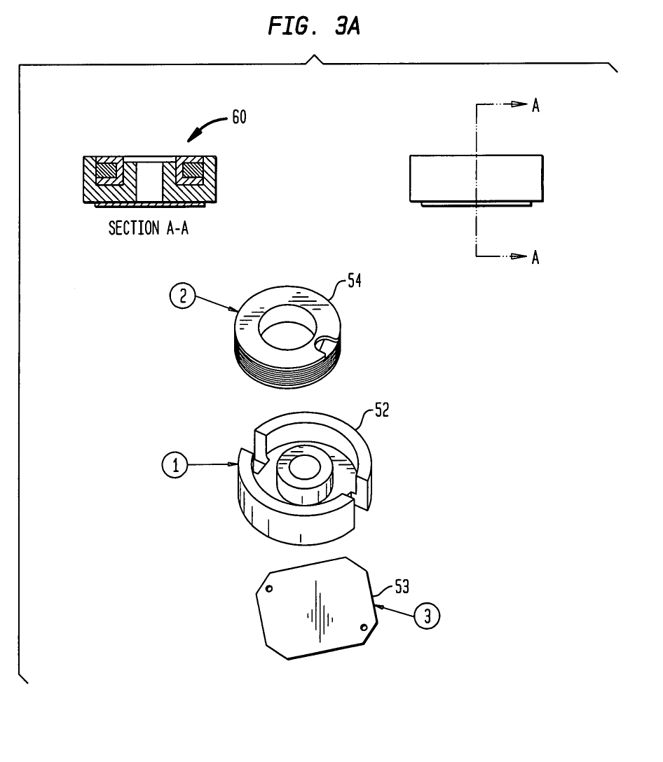

As discussed previously, the orthopedic instrumentation such as the reamer 22, handle 24 and tracker 28 contain electronic circuits. These circuits may be housed in packages that in turn are incorporated in various types of orthopedic instrumentation. An example of an embodiment of such package is shown in FIGS. 2-3.

FIG. 2 shows a package 50 that encloses a transponder that includes an electronic circuit including a RFID. FIG. 3 shows the package 50 placed in a cavity in an orthopedic instrument. The electronic circuit and the RFID need to be protected from contamination, impaction and sterilization. The protection of the electronic circuit and the RFID is achieved by encapsulating them using one of the various methods for encapsulation discussed hereafter.

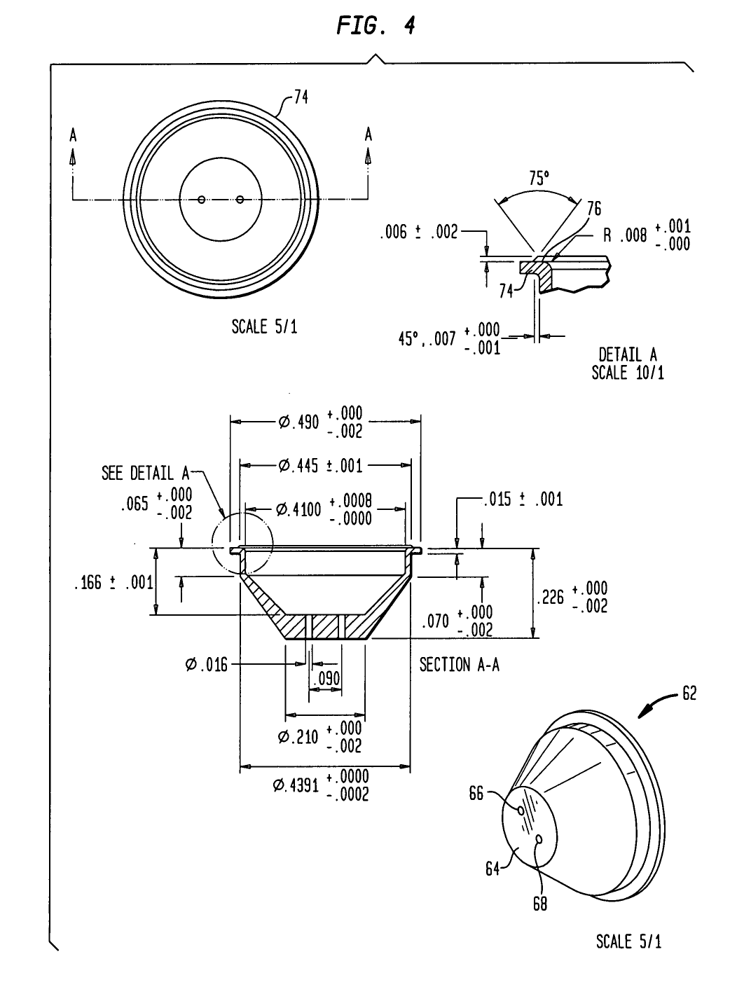

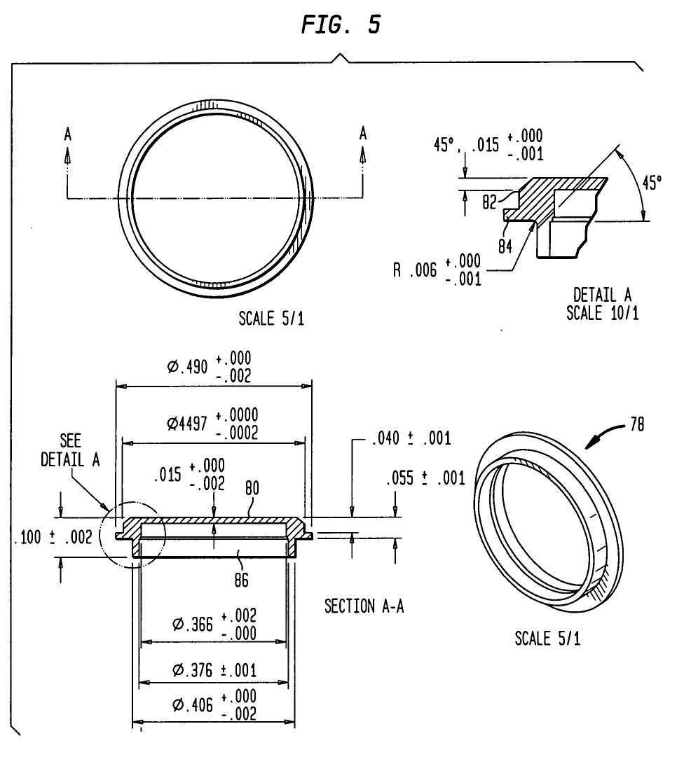

The package 50 may form a part of the orthopedic instrumentation, for example, the reamer 22, handle 24 or tracker 28. The package 50 includes a base 56, a cap 78 and a transponder 60. FIG. 3A shows details of transponder 60. Transponder 60 has a core 52, a flexible circuit 53 attached to the core 52 and a bobbin assembly 54 mounted in the core 52. FIG. 4 is a detailed drawing of the base 56. The base 56 is a cone shaped body 62 having a flat bottom surface 64. Two holes 66 and 68 are formed in the bottom surface 64. Holes 66 and 68 are optional and may be included or excluded in a different embodiment. Pins 70 and 72 (FIG. 2) pass from the interior of the body 62 to the exterior via holes 66 and 68. Pins 70 and 72 may be used to interconnect with other electronics on the orthopedic instrument. A lip 74 is formed at the opposing end from the bottom surface 64. A slight projection 76 is formed on the top surface of the lip 74. Projection 76 may aid in attaching a cap 78 (FIG. 5) to the base 56, for example, via welding or ultrasonic welding.

Cap 78 has a flat top surface 80 and a cylindrical wall 82 projecting from the top surface 80. A lip 84 is formed in approximately middle of wall 82. The bottom surface of lip 84 mates with the top surface of lip 74 when the cap 78 is assembled on the base 56. The assembly of the cap 78 and base 56 may be achieved by vibrating at ultrasonic frequency the cap 78 relative to the base 56 and melting the interface including the projection 76. Upon cooling of the interface, the cap 78 is welded to the base 56 forming a sealed interface. The cylindrical wall 82 forms a shallow cup 86 with the top surface 80 forming a base of the cup 86.

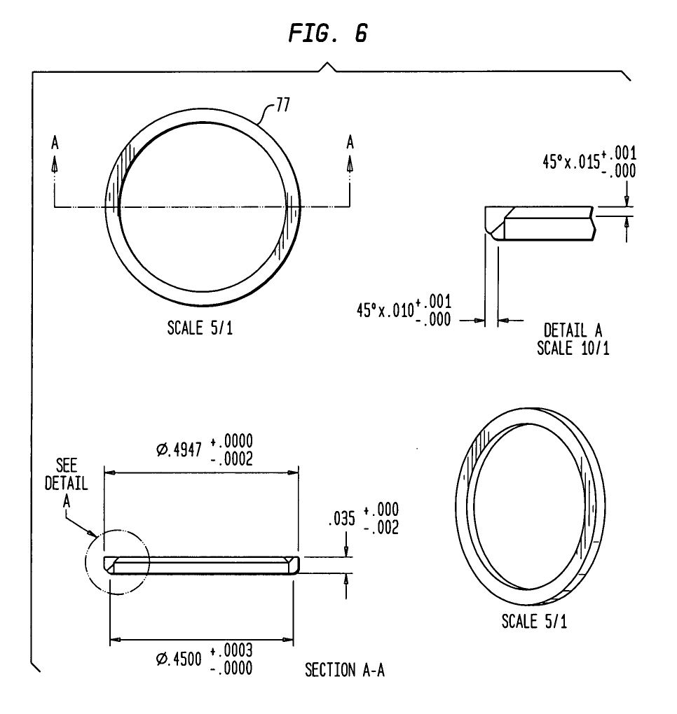

Prior to welding the base 56 to the cap 78, the transponder 60 is placed in the cup 86. The inside surface of the wall 82 and the external surface of cylindrical member 88 of the transponder 60 face each other with a small air gap between them. The transponder 60 is attached to the cap 78 by introducing suitable glue in the air gap. Cylindrical member 88 houses, inter alia, the transponder 60. The transponder 60 may be encapsulated in its attached position within the cap 78. Additionally, the entire package 50 may also be encapsulated in a cavity in the orthopedic instrumentation. Alternatively, the package 50 may be held in place by attaching a ring 77 (FIG. 6), for example, via welding to the mouth of the opening in the orthopedic instrument with the package 50 in place in the opening.

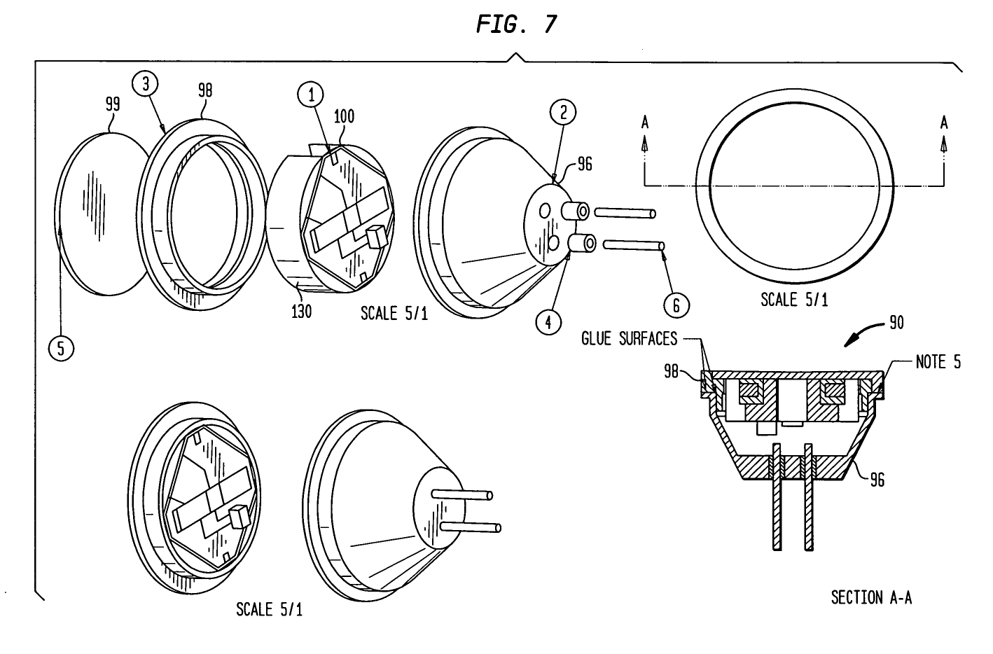

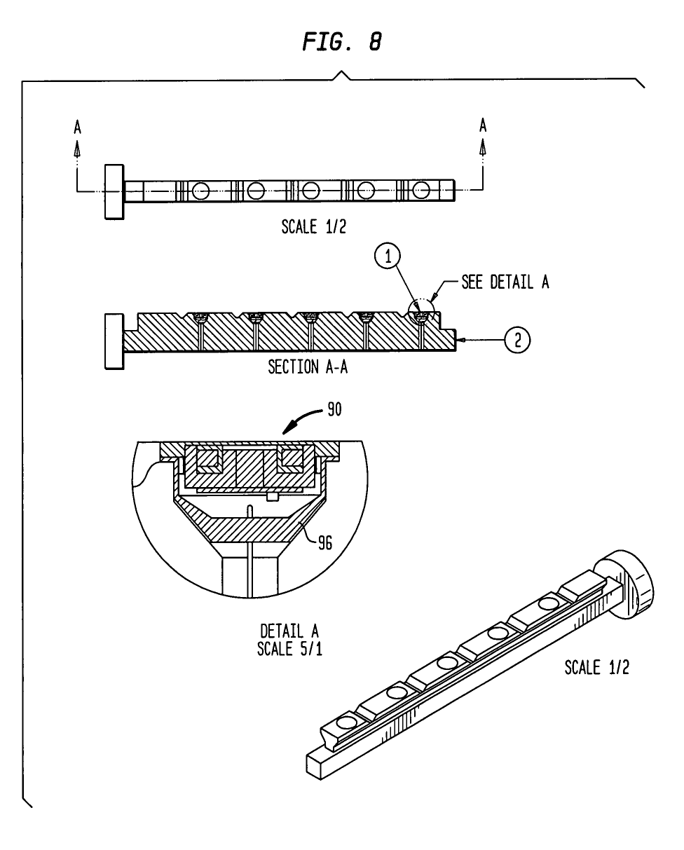

Another embodiment of a package that may house the transponder and may in turn be incorporated in various types of orthopedic instrumentation is shown in FIGS. 7-8. FIG. 7 shows a package 90 that encloses an electronic circuit including a RFID. FIG. 8 shows the package 90 placed in a cavity in an orthopedic instrument. The transponder 100 need to be protected from contamination, impaction and sterilization. The protection of the transponder 100 is achieved by encapsulating them using one of the various methods for encapsulation discussed hereafter.

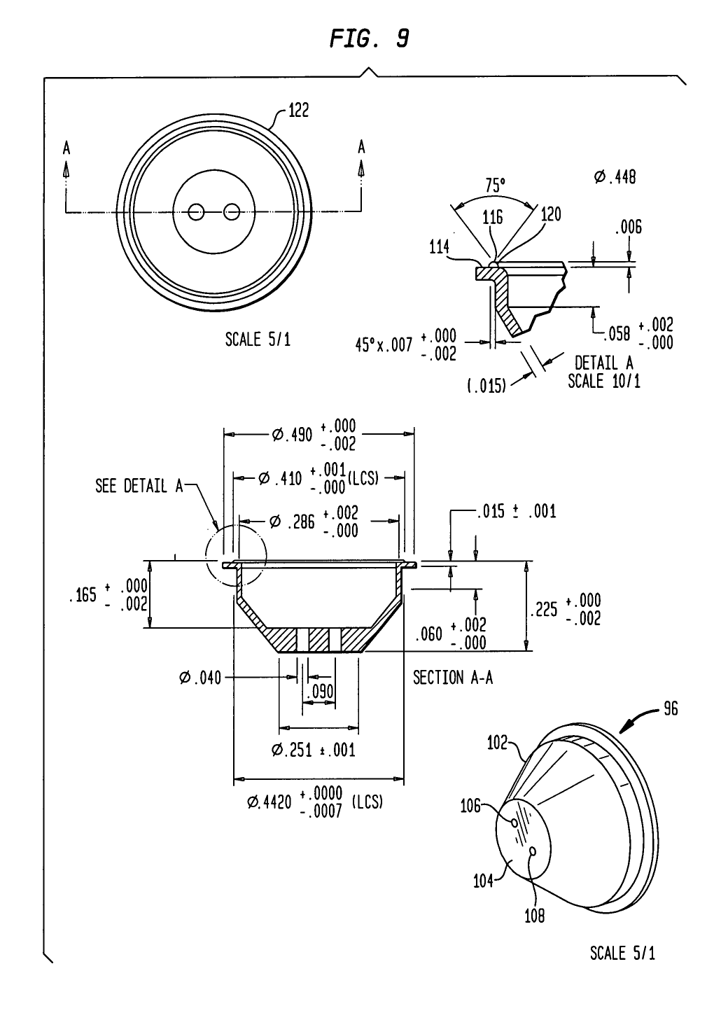

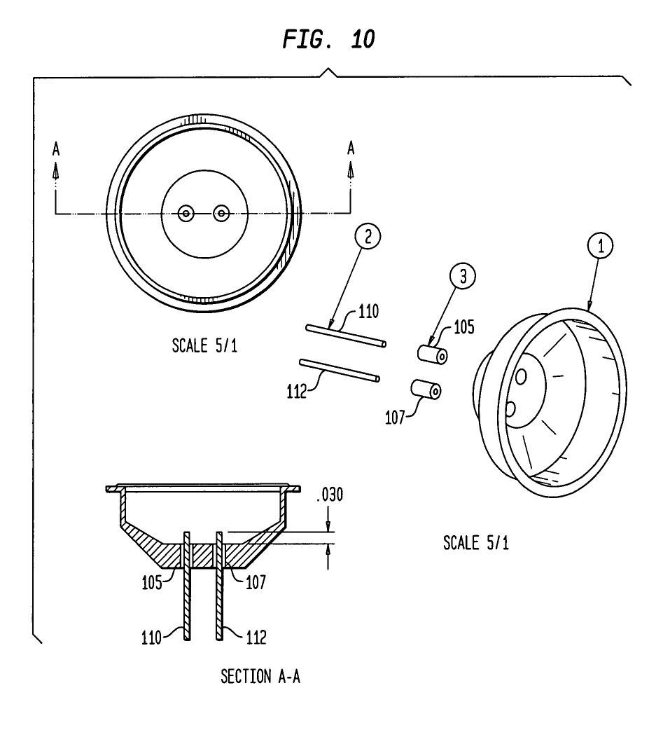

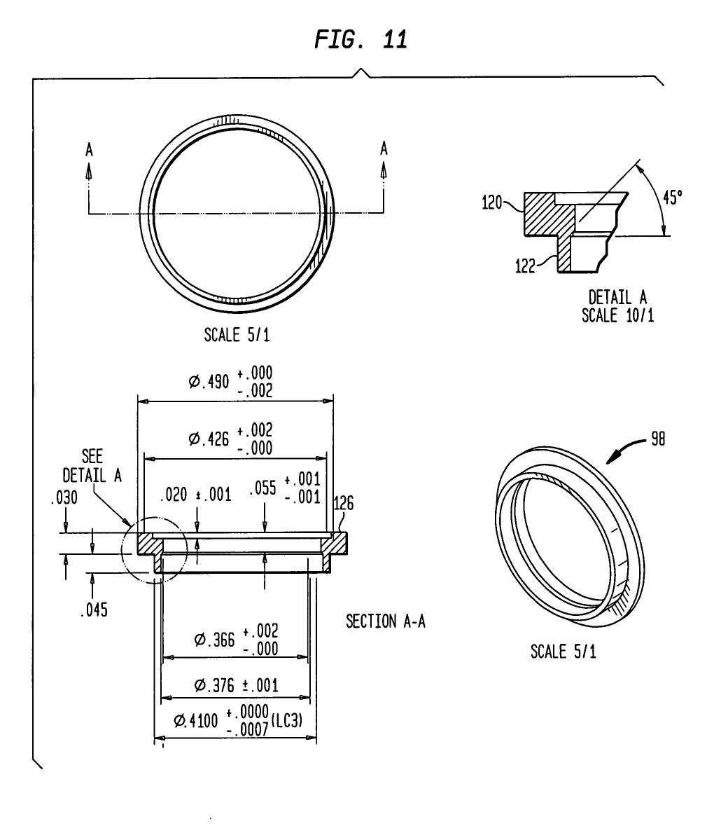

The package 90 may form a part of the orthopedic instrumentation, for example, the reamer 22, handle 24 or tracker 28. The package 90 includes a base 96, a metal cap 98, a non metallic lid 99 and the transponder 100. FIG. 9 is a detailed drawing of the base 96. The base 96 is a cone shaped body 102 having a flat bottom surface 104. Two holes 106 and 108 are formed in the bottom surface 104. Feed throughs 105 and 107 having holes in their center are inserted in holes 106 and 108 respectively. Pins 110 and 112 (FIG. 10) pass from the interior of the body 102 to the exterior via holes in the feed throughs 105 and 107 mounted in holes 106 and 108. Pins 110 and 112 may be used to interconnect with other electronics on the orthopedic instrument. A lip 114 is formed at the opposing end from the bottom surface 104. A slight projection 116 is formed on the top surface of the lip 114. FIG. 10 shows an assembly of the base 96, feed throughs 105 and 107 and pins 110 and 112. Projection 116 may aid in attaching a cap 98 (FIG. 11) to the base 96, for example, via welding or ultrasonic welding.

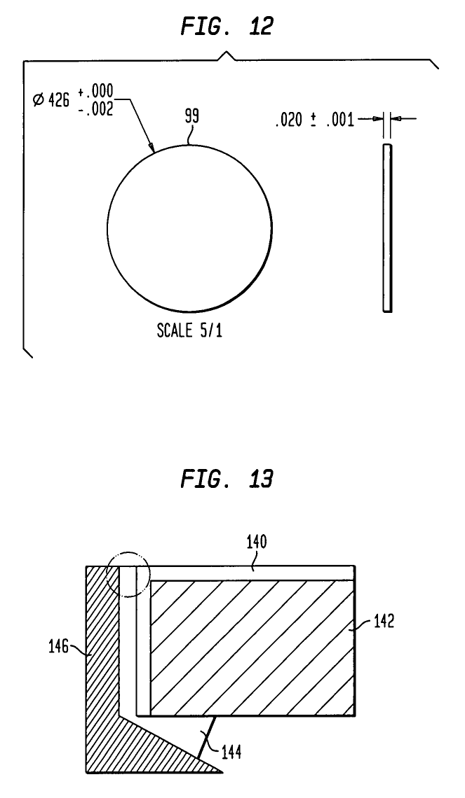

Cap 98 is generally cylindrical and has a larger cylindrical surface 120 and a smaller cylindrical surface 122. A lip surface 124 is formed at the juncture of cylindrical surface 120 and cylindrical surface 122. The lip surface 124 is perpendicular to the central axis of cylindrical surface 120 and cylindrical surface 122. The lip surface 124 mates with the top surface of lip 114 when the cap 98 is assembled on the base 96. The assembly of the cap 98 and base 96 may be achieved by vibrating at ultrasonic frequency the cap 98 relative to the base 96 and melting the interface including the projection 116. Upon cooling of the interface, the cap 98 is welded to the base 96 forming a sealed interface. Alternatively, the cap 98 may be welded on to the base 96. A sapphire glass lid 99 (FIG. 12) is attached on a ring shaped surface 126 formed near the top end of the cap 98. The lid 99 and cap 98 form a hermetic seal between them. The attachment may be achieved by applying glue to the mating surfaces or any other appropriate means including welding and ultrasonic welding. The cap 98 and lid 99, when assembled, form a shallow cup with the lid 99 forming a base of the cup.

Prior to welding the base 96 to the cap 98, the transponder 100 is placed in the cup formed by the cap 98 and the lid 99. The inside surface of the cylindrical portion 122 and the external surface of cylindrical member 130 of the transponder 100 face each other with a small air gap between them. The transponder 100 is attached to cap 98 by introducing suitable glue in the air gap. Cylindrical member 130 houses, inter alia, the transponder 100. The transponder 100 is encapsulated in its attached position within the cap 98.

The packages 50 and 90 of the above described exemplary embodiments may be encapsulated in a cavity in the orthopedic instrumentation using any one of the techniques discussed hereafter, or a combination of these techniques.

Encapsulation of Packages 50 and 90 and similar packages may be done using an Epoxy. Commercially available, autoclave resistant, epoxies such Masterbond EP42 HT-2, Zymet 505/515 and EpoTek 353ND may be used. Uncured epoxy may be formed to any shape due to its ability to flow and conform to complex geometries. The encapsulation of packages 50 and 90 using an Epoxy may be accomplished by one of the two encapsulation methods described below.

Method 1 (the molded method): When using the molded method, the transponder is placed/located into a mold cavity and uncured epoxy is injected around the assembly. The epoxy is then cured to maximize material properties for resistance to autoclave conditions. The molded assembly is placed in a cavity in the orthopedic instrument. Additional epoxy is then injected into the gap between the molded assembly and the instrument cavity and cured. The second epoxy application forms a seal between the encased electronics and instrument.

A variation of this method may comprise directly positioning the transponder in an appropriately sized cavity in the orthopedic instrument and uncured epoxy is injected around the transponder and then cured. This cured epoxy then fully encases the transponder were positioned in the instrument cavity. In this method the cup shaped cavity in the instrument serves as the mold for the epoxy.

Method 2 (the pre-formed method): This method is similar to the molded method above except in this scenario the epoxy is formed/molded into an appropriate shape and cured prior to it contacting transponder. After forming, the formed epoxy is then assembled onto the transponder or into the cavity in the orthopedic instrument to fabricate the necessary encapsulation geometry. Once assembled onto the transponder or into the cavity in the orthopedic instrument, the epoxy is re-cured to complete the encapsulation process.

Yet another technique is to use polymeric-epoxy combination. This technique utilizes a polymeric housing and epoxy, in combination, to provide sufficient protection from contamination, impaction and sterilization. The combination of materials allows for the polymeric material to provide additional resistance to gross contamination and the epoxy to provide the seal between polymer and metallic instrument. This method minimizes the use of epoxy.

FIG. 13 is a schematic diagram illustrating the method using the polymeric-epoxy combination. In this technique, a polymeric “cap 140” (transponder 60 or 100 described above may serve the function of the cap 140) is formed via either a machining or injection molding process. A transponder assembly (assembly 142 hereafter) is then assembled into the cap 140 and fixed in place through the use of an adhesive/epoxy. The cap 140 is then filled with an epoxy and cured to fully encase the assembly 142. This assembly 142 is then located in an appropriately sized cavity in the orthopedic instrument. Additional epoxy 144 is then injected into the gap between the assembly 142 and the cavity in the orthopedic instrument and cured. Epoxy 144 forms a seal between the assembly 142 and the orthopedic instrument.

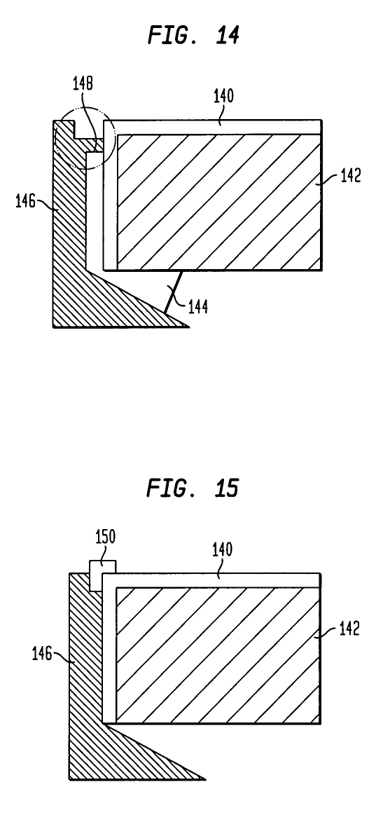

Additionally, to ensure a firm attachment between the assembly 142 and the cavity in the orthopedic instrument, there can be mechanical attachment between them. This mechanical attachment can be achieved through a variety of methods. One potential method involves “peening” the metallic material 146 that is adjacent to the epoxy 144 so that it comes into direct contact with the epoxy 144. See FIG. 14. The metal is deformed during this process, to form a tab 148 that mechanically fixes the assembly 142 in place. Another potential mechanical attachment method is to utilize a metallic ring 150 (FIG. 15) that contacts the polymeric cap 140 and the metallic material 146. Metallic ring 150 is typically made from stainless steel. Once assembled onto the orthopedic instrument, metallic ring 150 can be welded to mechanically fix the assembly 142 in place.

Yet another technique is to use metallic-epoxy combination. In this technique, a metallic “cap” is used in much the same way as the polymeric cap 140 described above. In this case, the “cap” is fabricated via machining and the assembly 142 is assembled into the “cap” in much the same manner as described above. Once this is accomplished, the “cap” can be attached to the orthopedic instrument either utilizing epoxy or metal ring as described above.

Yet another technique is to use all polymeric encapsulation. There are many polymeric materials that have been proven to be resistant to repeated autoclave cycles. Ultem® and Radel® are 2 examples of sterilization-resistant polymeric materials. These materials can be shaped into complex geometries via machining or molding processes making their utility in this application very appealing. The two polymeric components can be joined via several methods but the two most applicable methods are ultrasonic welding and laser welding.

In all polymeric technique, the base 56 and the cap 78 of FIG. 2 are made from Ultem or Radel via machining. The assembly 142 is inserted into the cap 78. The cap 78 is held in the base 56 via interference fit. Next the cap 59 or the base 56 is subjected to ultrasonic energy causing it to vibrate, while the other component is held motionless. The result is localized melting of the polymeric material at the interference point which, upon cooling, amalgamates to form a robust seal between the two components. This assembly is then inserted into the cavity in the orthopedic instrument and attached to the orthopedic instrument such as reamer 22, by welding a metal ring 150, as described previously, via laser welding.

Yet another technique is to use ceramic-metallic combination. In this case the ceramic material is typically glass or alumina material and the metallic components are typically stainless steel. The two materials can be joined in a variety of methods but the two most applicable methods, resistance welding and brazing. The Ceramic-Metallic encapsulation method is constructed from three separate components that are joined to form highly robust (“hermetic”) electronics housing.

The first of these components is a ceramic disc such as the non-metallic disc 99 of FIG. 12 that provides a pathway for the RF communication. In addition to being non-metallic, this item must be very thin in cross-section (less than 0.040″—to allow for RF communication) and also able to withstand impaction and repeated sterilization cycles. In this application, a glass (e.g., single crystal sapphire) or ceramic (e.g., Alumina) material can be utilized since they are available in thin cross-sections, can withstand the rigors of sterilization and most importantly, can be readily bonded or brazed onto stainless steel to form a highly robust seal between the two materials.

The second component in this assembly is a stainless steel “cap” such as cap 98 of FIG. 11 that serves as the intermediate part of the three piece housing. This component serves two purposes; first, it is used to attach the ceramic disc and second, it provides the connection point for the “base” component. Essentially this component is a flanged ring that is formed thorough conventional machining and includes features to accomplish both purposes mentioned above.

The third component is the “base” such as the base 96 of FIG. 9. This component can be made from a variety of materials but typically is made from a nickel-cobalt alloy (e.g., Kovar) due to its thermal expansion characteristics which allow it to bond efficiently with glass/ceramic materials used for feed-through 105 and 107. The geometry for this component can be formed using conventional machining. After the electronics assembly 142 is assembled and bonded to the ceramic disc (or glass disc) and “cap” assembly, this component is then resistance welded to the “base” to complete the housing.

Although the invention herein has been described with reference to particular embodiments, it is to be understood that these embodiments are merely illustrative of the principles and applications of the present invention. For example, the invention described herein may also be applied to rotating couplings. It is therefore to be understood that numerous modifications may be made to the illustrative embodiments and that other arrangements may be devised without departing from the spirit and scope of the present invention as defined by the appended claims.

I claim:

1. A surgical tool, comprising:
a body having a cavity;
an RFID electronic assembly, the RFID electronic assembly having a storage element connected to an antenna and operable to transmit information associated with the surgical tool;
a first layer of first encapsulant formed around the RFID electronic assembly;
a housing insertable into the cavity, the encapsulated RFID electronic assembly being mounted within the housing; and
a second layer of second encapsulant formed around the housing to hold it in place in the body cavity.

2. The surgical tool of claim 1, wherein the surgical tool comprises a cutting accessory selected from the group consisting of a reamer, a burr and a broach.

3. The surgical tool of claim 1, wherein the surgical tool comprises a handpiece.

4. The surgical tool of claim 1, wherein the first and second encapsulants comprise an epoxy resin.

5. The surgical tool of claim 1, wherein the first encapsulant comprises a polymer having a predetermined amount of particles that are operable to reduce the growth rate of the polymer.

6. The surgical tool of claim 1, further comprising mechanically affixing the housing to the cavity.

7. The surgical tool of claim 6, wherein mechanical affixing comprises peening a metallic base over the second layer of second encapsulant formed around the housing.

8. The surgical tool of claim 1, wherein the cavity terminates as an opening on a surface of the surgical tool and the housing comprises a base and a cap mounted onto the base, the housing being located in the cavity.

9. The surgical tool of claim 8, further comprising a metallic ring positioned adjacent the cap and the surface of the surgical tool and operable to secure the housing in place.

10. The surgical tool of claim 9, wherein the metallic ring comprises a stainless steel ring.

11. The surgical tool of claim 1, wherein the housing is made from polymeric material selected from the group consisting of Ultem and Radel.

12. An orthopedic instrument, comprising
a body having a cavity;
a housing located within the cavity, the housing comprising a base member, a cap attached to the base and a glass disc attached to the cap, the glass disk being adapted to allow radio frequency waves to pass through its body; and
an RFID electronic assembly securably located within the cavity, the RFID electronic assembly being encapsulated using a first potting compound.

13. The orthopedic instrument of claim 12, further comprising:
a second potting compound, the second potting compound encapsulating the housing in the cavity to provide protection for the RFID electronic assembly from contamination, impaction and sterilization.

14. The orthopedic instrument of claim 13, wherein the cavity terminates as an opening on a surface of the orthopedic instrument and the cap member is located adjacent the surface in the opening.

15. The orthopedic instrument of claim 14, wherein the disc is made from a ceramic.

16. The orthopedic instrument of claim 15, further comprising a stainless steel ring mounted adjacent to the cap member.

17. The orthopedic instrument of claim 16, wherein the cap and base members are resistance welded to each other.

18. The orthopedic instrument of claim 16, wherein the cap and base members are brazed with each other.

19. The orthopedic instrument of claim 18, wherein the RFID electronic assembly comprises a storage element connected to an antenna and operable to transmit information associated with the orthopedic instrument.
